(19) United States
(12) Patent Application Publication
(10) Pub. No.: US 2017/0370861 A1
(43) Pub. Date: Dec. 28, 2017

(54) APPARATUS AND METHOD FOR DETECTING EXPLOSIVES

(71) Applicant: ONE RESONANCE SENSORS, LLC, San Diego, CA (US)

(72) Inventors: Pablo J. PRADO, San Diego, CA (US); Timothy RAYNER, (US)

(21) Appl. No.: 15/539,098

(22) PCT Filed: Dec. 17, 2015
(86) PCT No.: PCT/US2015/066495

§ 371 Date: Jun. 22, 2017

Related U.S. Application Data
(60) Provisional application No. 62/096,358, filed on Dec. 23, 2014.

(51) Int. Cl. G01N 24/08; G01R 33/44; G01N 33/22

(52) U.S. Cl. CPC G01N 24/084; G01N 33/227; G01R 33/441

(57) ABSTRACT

Portable electronic devices may be inspected for the presence of explosives using a combination of nuclear quadrupole resonance (NQR) and explosive trace detection (ETD). NQR may be used to detect bulk or sheet explosives while the ETD may be used to detect minute quantities of explosive particulates. An alarm indication may be generated when either the NQR spectroscopy or the ETD detects an explosive material.

### BACKGROUND

### Field of the Invention

The present invention is generally related to the detection of explosives and is more specifically related to the detection of explosives using a combination of nuclear quadrupole resonance (NQR) spectroscopy and explosive trace detection (ETD).

### Related Art

Hidden explosives pose a significant and well-documented threat to public safety. Mass transit systems, particularly commercial airliners, have been perpetual targets for acts of terrorism. Over the last three decades, the extent of passenger and luggage screening has drastically increased in response to atrocities like the bombing of Pan Am Flight 103 and the September 11 attacks. But while some of the more recent attempts to smuggle explosives onboard aircrafts have been crude, security experts anticipate that the next iteration of improvised explosive devices (IEDs) to emerge will be more sophisticated, diverse, and clandestine. In particular, stealthy IEDs may masquerade as common portable consumer electronic devices (e.g., smartphones, tablet PCs).

But current screening technologies are able to account for a limited array of explosive materials, whereas a gamut of explosives may be smuggled under clever guises through security checkpoints. X-Rays, for example, do not provide sufficient spatial resolution to enable a thorough inspection of small compartments and cavities. In particular, explosive materials that have been arranged in a sheet or planar configuration inside, for example, an iPhone® or an iPad®, will generally appear innocuous in an X-Ray scan. Meanwhile, some ETD techniques cannot detect explosives having low vapor pressure. Thus, IEDs that have been hermetically sealed will generally be able to evade detection by ETD. Other ETD techniques may rely on the presence of particulates. Consequently, cleaning the exterior surface of an TED will effectively frustrate the ability to use ETD to accurately identify the TED as a threat.

In addition, optical techniques (e.g., spatially offset Raman spectroscopy (SORS)) can be easily foiled by opaque cases, containers, or packaging. Finally, even NQR spectroscopy lacks the ability to detect every type of explosive materials.

### SUMMARY

To effectively and efficiently detect a broad range of explosives, various embodiments of the apparatus and method described herein are directed toward using a combination of NQR spectroscopy and ETD to detect explosive compounds, substances, or materials that have been deliberately embedded, camouflaged, or otherwise concealed within various objects. For example, NQR spectroscopy and ETD may be used in combination to detect explosives that are hidden within personal or portable electronic devices, including, for example, but not limited to, smartphones, tablet PCs, laptops, and headsets.

In some embodiments, the NQR and ETD sensors may be physically integrated within a single apparatus. Meanwhile, NQR spectroscopy and one or more ETD techniques may be applied simultaneously or in sequence.

Other features and advantages of the present invention will become more readily apparent to those of ordinary skill in the art after reviewing the following detailed description and accompanying drawings.

### BRIEF DESCRIPTION OF THE DRAWINGS

Aspects and features of the present inventive concept will be more apparent by describing example embodiments with reference to the accompanying drawings, in which:

FIG. 1A illustrates a configuration of an apparatus according to various embodiments;

FIG. 1B illustrates a configuration of an apparatus according to various embodiments;

FIG. 2A illustrates a configuration of an apparatus according to various embodiments;

FIG. 2B illustrates a configuration of an apparatus according to various embodiments;

FIG. 3A is a flowchart illustrating a process for detecting explosives according to various embodiments;

FIG. 3B is a flowchart illustrating a process for detecting explosives according to various embodiments; and

FIG. 4 illustrates a wired or wireless processor enabled device according to various embodiments.

### DETAILED DESCRIPTION

Certain embodiments disclosed herein provide for an apparatus and a method of detecting concealed explosives. For example, in various embodiments, the apparatus may combine and/or integrate an NQR sensor and an ETD sensor. In various embodiments, the method may include a sequential and/or simultaneous performance of one or more instances of both NQR spectroscopy and ETD. After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

In various embodiments, NQR spectroscopy may be used in the bulk detection of explosive compounds, substances, or materials. NQR spectroscopy is a chemical analysis technique that exploits the electric quadrupole moment possessed by certain atomic nuclei (e.g., ¹⁴N, ¹⁷O, ³⁵Cl, and ⁶³Cu). An electric quadrupole moment arises from the presence of two adjacent electric dipoles (i.e., opposite charges separated by a short distance) in an atomic nucleus. Otherwise stated, an electric quadrupole moment is caused by an asymmetry in the distribution of the positive electric charge within the nucleus, which is typically the case for any atomic nucleus described as either a prolate (i.e., “stretched”) or oblate (i.e., “squashed”) spheroid.

The interaction between the intrinsic electric quadrupole moment and an electric field gradient (EFG) within the nucleus generates distinct energy states. As such, the primary goal of NQR spectroscopy is to determine the resonant or NQR frequency at which the transition between these distinct energy states occur and then relate this property to a specific material, substance, or compound. Since the EFG surrounding a nucleus in a given substance is determined primarily by the valence electrons engaged in the formation of chemical bonds with adjacent nuclei, different substances will exhibit distinct resonant or NQR frequencies. The NQR frequency of a substance depends on both the nature of each atom comprising the substance and on the overall chemical environment (i.e., the other atoms in the substance). This renders NQR spectroscopy especially sensitive to the chemistry or composition of each substance.

When a substance is irradiated or interrogated with radio frequency (RF) electromagnetic radiation, energy will be absorbed by each nucleus within the substance when the frequency of the interrogation electromagnetic radiation coincides with the specific NQR frequency for that substance. The absorption of energy at the specific NQR frequency for the substance causes a transition to a higher energy state followed by an emission of energy (i.e., feedback electromagnetic radiation) during a subsequent return to a lower energy state. This emission of energy is at the same frequency as the NQR frequency specific to that substance. As such, the NQR frequency of the feedback electromagnetic radiation emitted by a substance can act as a chemical signature for that substance. With respect to explosives, the NQR frequency of one or more chemical components of an explosive substance, material, or compound can be used to identify the presence of the explosive regardless of efforts to physically conceal the explosives, such as within an electronic device.

In various embodiments, ETD may be used to detect trace quantities of explosive compounds, substances, or materials. To detect small amounts of explosives, ETD may rely on explosive vapor detection and/or particulate sampling. In various embodiments, appropriate or applicable ETD techniques may include, for example, but not limited to, ion mobility spectroscopy (IMS), thermo redox, chemiluminescence, amplifying fluorescent polymer (APF), and mass spectrometry (MS).

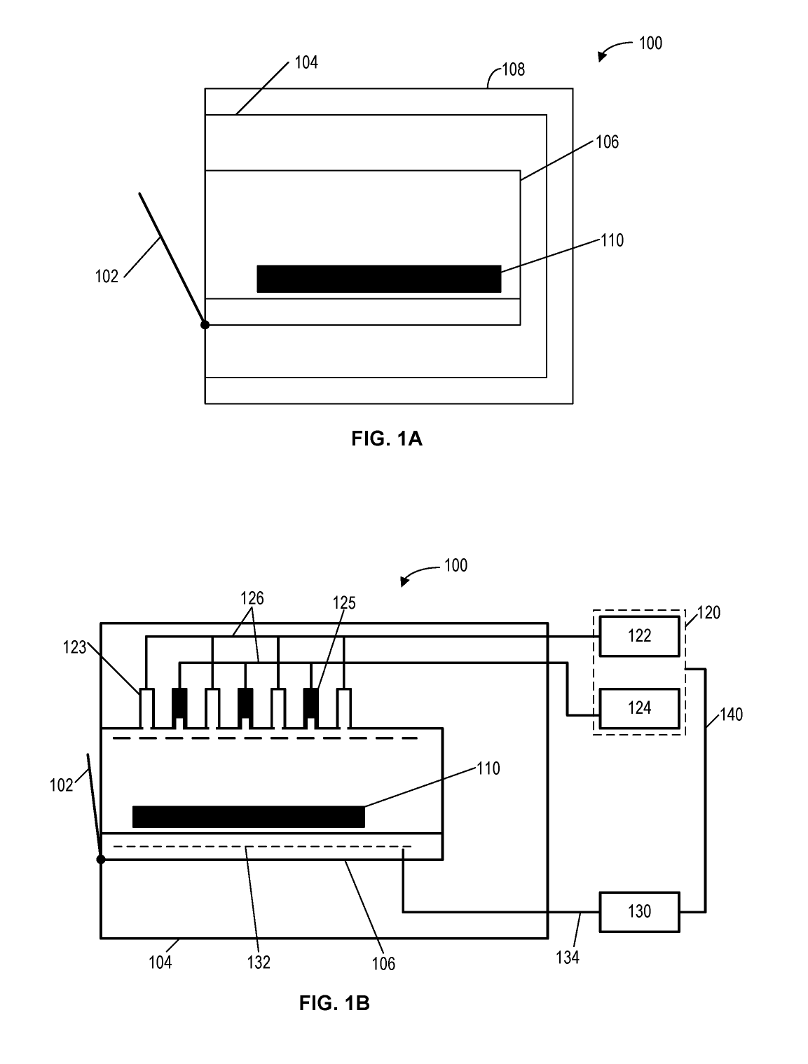

FIG. 1A illustrates a configuration of an apparatus 100 according to various embodiments. Referring to FIG. 1A, the apparatus 100 may include a door 102, an RF shield 104, an air containment chamber 106, and an enclosure 108. In some embodiments, an inspected object 110 may be placed directly into the air containment chamber 106 inside the apparatus 100. The inspected object 110 may be a suspected IED including, for example, but not limited to a personal or portable electronic device such as a smartphone, tablet PC, and laptop. The door 102 may open to reveal and provide access into the air containment chamber 106. In some embodiments, the door 102 and the air containment chamber 106 may create a hermetically sealed environment that enhances the efficacy of ETD.

In various embodiments, the enclosure 108 may enclose or surround the RF shield 104. Meanwhile, the RF shield 104 may be an intermediary layer between the enclosure 108 and the air containment chamber 106. In various embodiments, the RF shield 104 may enhance the efficacy of NQR spectroscopy by minimizing interference and noise signals from the surrounding environment.

FIG. 1B illustrates a configuration of an apparatus 100 according to various embodiments. Referring to FIG. 1B, the apparatus 100 may further include or be coupled to an ETD system 120 (i.e., trace/vapor detection) that is configured to detect explosive compounds, substances, or materials using ETD.

The ETD system 120 may include an air sampling unit 122 and a synchronized intermittent pump 124. The ETD system 120 may further include one or more pipes 126. The one or more pipes 126 may be coupled to the ETD system 120, for example, to the air sampling unit 122 and the synchronized intermittent pump 124. Moreover, the one or more pipes 126 from the air sampling unit 122 may be fitted with one or more air sampling nozzles 123. The air sampling nozzles 123 may be installed, in an airtight manner, over apertures in the air containment chamber 106. The one or more pipes 126 from the synchronized intermittent pump 124 may also be fitted with one or more blowing nozzles 125. The one or more blowing nozzles 125 may be installed over apertures in the air containment chamber 106 in a same, similar, or different manner as the air sampling nozzles 123.

In various embodiments, the one or more blowing nozzles 125 may be configured to inject one or more gaseous substances (e.g., air) from the synchronized intermittent pump 124 into the air containment chamber 106. As a result, the inspected object 110 may be exposed to the one or more gaseous substances. The one or more air sampling nozzles 123 may be configured to extract one or more gaseous substances (e.g., air) from the air containment chamber 106. The air sampling unit 122 may analyze or inspect the gaseous substances from the air containment chamber 106 to determine whether one or more explosive compounds, substances, or materials are present in the inspected object 110. For example, after the inspected object 110 is exposed to the one or more gaseous substances introduced into the air containment chamber 106 by the synchronized intermittent pump 124, the air sampling unit 122 may analyze and inspect gaseous substances extracted from the air containment chamber 106. The ETD system 120 may display the results of the ETD, including any alarm indications in the event that the analysis and inspection of the gaseous substances extracted from the air containment chamber indicates a presence of an explosive compound, material, or substance.

In various embodiments, the apparatus 100 may further include or be coupled to an NQR system 130 (i.e., quadrupole resonance RF system) that is configured to detect explosive compounds, substances, or materials using NQR spectroscopy. The NQR system 130 may include an RF antenna 132 that is coupled to an RF input/output 134. As will be described in more detail below, the ETD system 120 and the NQR system 130 may be coupled to and integrated with the apparatus 100 in a variety of configurations. For example, the NQR system 130 may operate as a master system and is native to the apparatus 100 while the ETD system 120 may be a secondary system that is later attached to the apparatus 100. In various embodiments, the ETD system 120 and the NQR system 130 may be coupled via a connection 140. In various embodiments, the connection 140 may be a wired or wireless communication link.

In various embodiments, once placed inside the apparatus 100, the inspected object 110 may be subject to a sequence of specifically timed interrogation electromagnetic radiation from the NQR system 130. Moreover, the NQR system 130 may measure the frequencies of the feedback electromagnetic radiation emitted by the inspected object 110 in response to the interrogation magnetic radiation. The NQR system 130 may determine whether the frequencies of the feedback electromagnetic radiation correspond to NQR frequencies that uniquely identify explosive compound, substances, or materials. In some embodiments, the NQR system 130 may display the results of the NQR spectroscopy, including any alarm indications in the event that the frequency of the feedback electromagnetic radiation indicates the presence of an explosive compound, substance, or material.

In some embodiments, the apparatus 100 may be configured with the RF antenna 132 inside the air containment chamber 106. In those embodiments, the RF antenna 132 may be configured to permit at least one of an entry of one or more gaseous substances into the air containment chamber 106 via the blowing nozzles 125 and an exit of one or more gaseous substances from the air containment chamber 106 via the air-sampling nozzles 123. Alternately, in other embodiments, the RF antenna 132 may be disposed outside of the air containment chamber 106.

Although not shown in FIG. 1A or 1B, in some embodiments, the apparatus 100 may further include a conveyor system. The conveyor system may be integrated with the door 102 and the air containment chamber 106 in a manner that allows the air containment chamber 106 to provide a hermetically sealed environment. For example, the inspected object 110 may be placed on the conveyor system at an entrance of the air containment chamber 106. The conveyor system may transported into the apparatus 110 and over a length of the air containment chamber 106, while ensuring appropriate exposure to interrogation electromagnetic radiation from the NQR system 130 and/or gaseous substances from the ETD system 120.

Although the NQR system 130 and the ETD system 120 are shown as individual components of the apparatus 100 in FIG. 1B, a person having ordinary skill in the art can appreciate that apparatus 100 may be modular and exhibit different configurations without departing from the scope of the present inventive concept. In one embodiment, the apparatus 100 may be an NQR system that includes a portion of the ETD system 120 shown in FIG. 1A. For example, the apparatus 100 may be an NQR system that provides the air sampling nozzles 123, the blowing nozzles 125, and the one or more pipes 126 shown in FIG. 1A. The apparatus 100 may further include a valve or an inlet (not shown). As such, any original equipment manufacturer (OEM) ETD system may be later coupled to and integrated with the apparatus 100 via the valve or the inlet. The apparatus 100 may be adaptable to interface with and to control the OEM ETD system such that the OEM ETD system may operate under the control of the NQR system. The apparatus 100 may display results from the NQR system and may be further adaptable to also display results from the OEM ETD system.

In an alternate embodiment, the apparatus 100 may be an ETD system that includes a portion of the NQR system shown in FIG. 1A. For example, the apparatus 100 may be an ETD system that includes the RF antenna 132 and the RF input/output 134. In such an embodiment, any OEM NQR system may be coupled to and integrated with the apparatus 100. The apparatus 100 may be adaptable to interface with and to control the OEM NQR system. The apparatus 100 may further be configured to display results from both the ETD and the OEM NQR system.

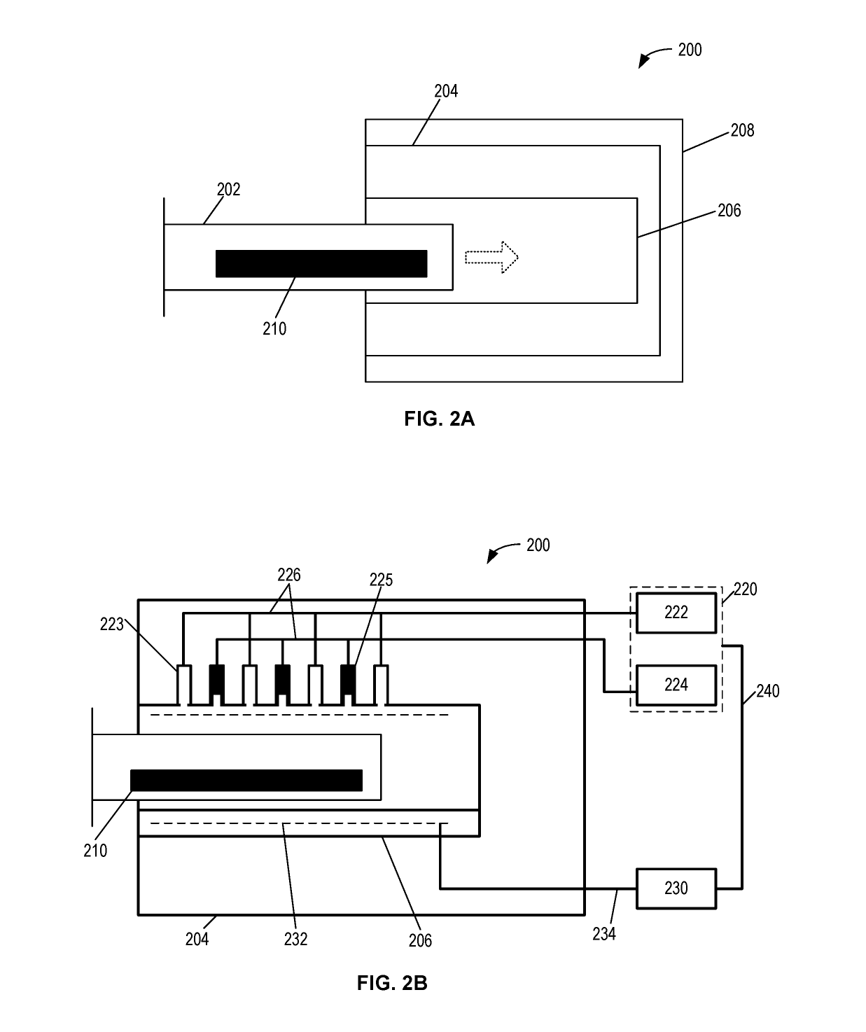

FIG. 2A illustrates a configuration of an apparatus 200 according to various embodiments. With reference to FIG. 2A, in some embodiments, the apparatus 200 includes a tray 202 that is configured to slide in and out of an air containment 206. Instead of placing an inspected object 210 directly into the air containment 206, the inspected object 210 may be placed inside the tray 202 and slid inside the apparatus 200. In some embodiments, the tray 202 in a closed position and the air containment 206 may create a hermetically sealed environment inside the apparatus 200 that enhances the efficacy of ETD.

In various embodiments, the apparatus 200 may further include an RF shield 204 and an enclosure 208. The enclosure 208 may enclose or surround the RF shield 204. Meanwhile, the RF shield 204 may be an intermediary layer between the enclosure 208 and the air containment 206. In various embodiments, the RF shield 204 may enhance the efficacy of NQR spectroscopy by minimizing interference and noise signals from the surrounding environment.

FIG. 2B illustrates a configuration of an apparatus 200 according to various embodiments. With reference to FIGS. 2A and 2B, in various embodiments, the apparatus 200 may include the tray 202, which may be used to insert the inspected object 210 inside the apparatus 200.

In various embodiments, the apparatus 200 further includes an ETD system 220 (i.e., trace/vapor detection) and an NQR system 230 (i.e., quadrupole resonance RF system). The ETD system 220 and the NQR system 230 may be coupled via a connection 240. In various embodiments, the connection 240 may be a wired or wireless communication link.

The ETD system 220 may include an air sampling unit 222 and a synchronized intermittent pump 224 that may both be coupled to one or more pipes 226. The end of each of the one or more pipes may be fitted with an air sampling nozzle 223 or a blowing nozzle 225. One or more air sampling nozzles 223 and blowing nozzles 225 may be installed, in an airtight manner, over apertures in the air containment chamber 206. In some embodiments, depending on the distance between the air containment chamber 206 and the ETD system 220, the one or more pipes 226 may be subject to one or more treatments. For example, in one embodiment, the one or more pipes 226 may be heated.

The NQR system 230 may include an RF antenna 232 and an RF input/output 234. In some embodiments, the RF antenna 232 may be placed inside the air containment 206 and may be configured to permit an entry of one or more gaseous substances into the air containment 206 and/or an exit of one or more gaseous substances out of the air containment 206.

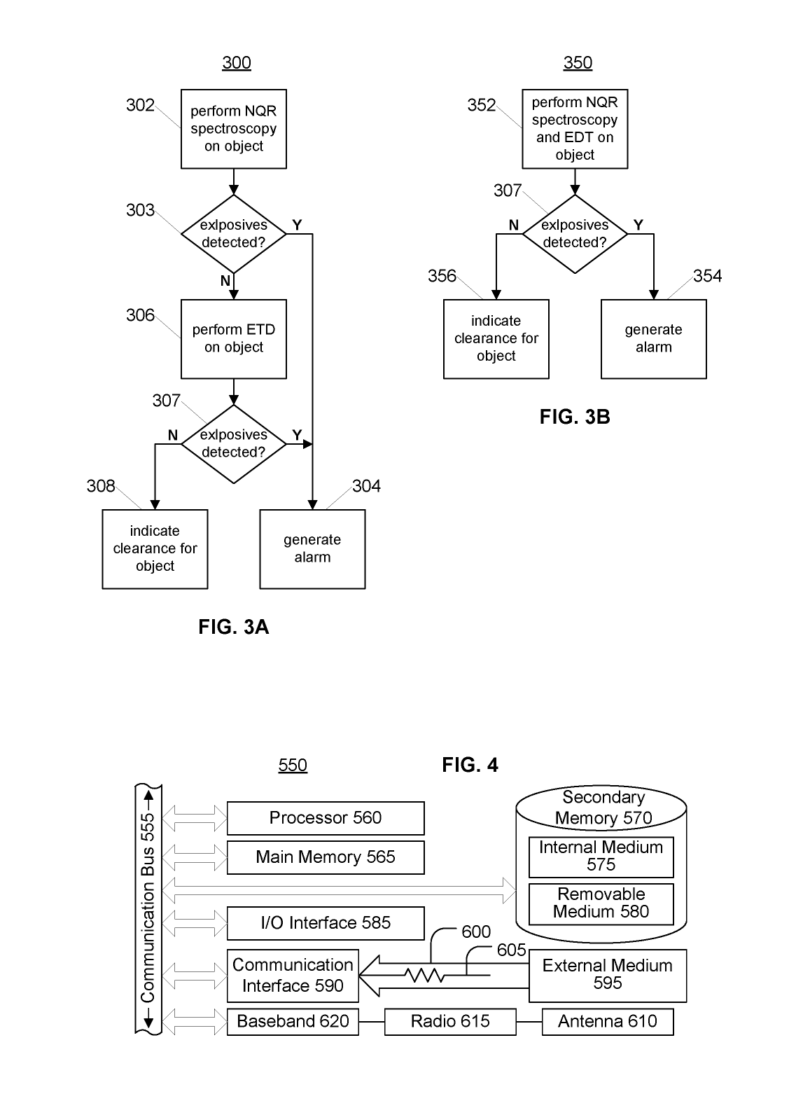

FIG. 3A illustrates a process 300 according to various embodiments. Referring to FIGS. 1A, 1B, 2A, 2B, and 3A, in various embodiments, the process 300 may be performed by the apparatus 100 or the apparatus 200 described with respect to FIGS. 1A and 1B, and 2A and 2B. In some embodiments, NQR spectroscopy and ETD may be performed sequentially.

NQR spectroscopy may be performed on an object (302). If one or more explosive compounds, substances, or materials are detected as a result of the NQR spectroscopy (303-Y), an alarm may be generated (304). Alternately, if one or more explosive compounds, substances, or materials are not detected as a result of the NQR spectroscopy (303-N), ETD may be performed on object. If the ETD detects one or more explosive compounds, substances, or materials (307-Y), an alarm may be generated (304). Alternately, if the ETD does not detect one or more explosive compounds, substances, or materials (307-N), clearance may be indicated for the object (308).

As shown in FIG. 3A, NQR spectroscopy may be performed before ETD in the process 300. However, a person having ordinary skill in the art can appreciate that NQR spectroscopy and ETD may be performed in any order without departing from the scope of the present inventive concept. Furthermore, for clarity and convenience, the process 300 includes a single occurrence each of NQR spectroscopy and ETD. But a person having ordinary skill in the art can appreciate that NQR spectroscopy and/or ETD may be repeated any appropriate, desired, or required number of times without departing from the scope of the present inventive concept. In some embodiments, between successive instances of ETD, the one or more pipes 226 may be subject to one or more cleaning treatments. For example, the one or more pipes 226 may be heated after one instance of ETD is completed and before the next instances of ETD.

FIG. 3B illustrates a process 350 according to various embodiments. Referring to FIGS. 1A, 1B, 2A, 2B, and 3B, in various embodiments, the process 350 may be performed by the apparatus 100 or the apparatus 200 described with respect to FIGS. 1A and 1B, and 2A and 2B. In some embodiments, NQR spectroscopy and ETD may be performed simultaneously or in parallel.

Both NQR spectroscopy and ETD may be performed at the same time or in parallel on an object (352). If either the NQR spectroscopy or the ETD detects one or more explosive compounds, substances, or materials (353-Y), an alarm may be generated (354). Alternately, if neither the NQR spectroscopy nor the ETD detects one or more explosive compounds, substances, or materials (353-N), clearance may be indicated for the object (356).

For clarity and convenience, the process 350 includes a single occurrence each of NQR spectroscopy and ETD. But a person having ordinary skill in the art can appreciate that NQR spectroscopy and/or ETD may be repeated any appropriate, desired, or required number of times without departing from the scope of the present inventive concept. Furthermore, some instances of NQR spectroscopy and ETD may be performed simultaneously or in parallel, while other instances may be performed sequentially in any order.

FIG. 4 illustrates a wired or wireless system 550 according to various embodiments. With reference to FIGS. 1A, 1B, 2A, 2B, 3A and 3B, in various embodiments, the system 550 may be used to implement various controller modules comprising the apparatus 100 or the apparatus 200 described with respect to FIGS. 1A and 1B, and 2A and 2B. The system 550 can be a conventional personal computer, computer server, personal digital assistant, smart phone, tablet computer, or any other processor enabled device that is capable of wired or wireless data communication. Other computer systems and/or architectures may be also used, as will be clear to those skilled in the art.

System 550 preferably includes one or more processors, such as processor 560. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 560.

The processor 560 is preferably connected to a communication bus 555. The communication bus 555 may include a data channel for facilitating information transfer between storage and other peripheral components of the system 550. The communication bus 555 further may provide a set of signals used for communication with the processor 560, including a data bus, address bus, and control bus (not shown). The communication bus 555 may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPIB”), IEEE 696/S-100, and the like.

System 550 preferably includes a main memory 565 and may also include a secondary memory 570. The main memory 565 provides storage of instructions and data for programs executing on the processor 560. The main memory 565 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

The secondary memory 570 may optionally include a internal memory 575 and/or a removable medium 580, for example a floppy disk drive, a magnetic tape drive, a compact disc (“CD”) drive, a digital versatile disc (“DVD”) drive, etc. The removable medium 580 is read from and/or written to in a well-known manner. Removable storage medium 580 may be, for example, a floppy disk, magnetic tape, CD, DVD, SD card, etc.

The removable storage medium 580 is a non-transitory computer readable medium having stored thereon computer executable code (i.e., software) and/or data. The computer software or data stored on the removable storage medium 580 is read into the system 550 for execution by the processor 560.

In alternative embodiments, secondary memory 570 may include other similar means for allowing computer programs or other data or instructions to be loaded into the system 550. Such means may include, for example, an external storage medium 595 and an interface 570. Examples of external storage medium 595 may include an external hard disk drive or an external optical drive, or and external magneto-optical drive.

Other examples of secondary memory 570 may include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage media 580 and communication interface 590, which allow software and data to be transferred from an external medium 595 to the system 550.

System 550 may also include an input/output (“I/O”) interface 585. The I/O interface 585 facilitates input from and output to external devices. For example the I/O interface 585 may receive input from a keyboard or mouse and may provide output to a display. The I/O interface 585 is capable of facilitating input from and output to various alternative types of human interface and machine interface devices alike.

System 550 may also include a communication interface 590. The communication interface 590 allows software and data to be transferred between system 550 and external devices (e.g. printers), networks, or information sources. For example, computer software or executable code may be transferred to system 550 from a network server via communication interface 590. Examples of communication interface 590 include a modem, a network interface card (“NIC”), a wireless data card, a communications port, a PCMCIA slot and card, an infrared interface, and an IEEE 1394 fire-wire, just to name a few.

Communication interface 590 preferably implements industry promulgated protocol standards, such as Ethernet IEEE 802 standards, Fiber Channel, digital subscriber line (“DSL”), asynchronous digital subscriber line (“ADSL”), frame relay, asynchronous transfer mode (“ATM”), integrated digital services network (“ISDN”), personal communications services (“PCS”), transmission control protocol/Internet protocol (“TCP/IP”), serial line Internet protocol/point to point protocol (“SLIP/PPP”), and so on, but may also implement customized or non-standard interface protocols as well.

Software and data transferred via communication interface 590 are generally in the form of electrical communication signals 605. These signals 605 are preferably provided to communication interface 590 via a communication channel 600. In one embodiment, the communication channel 600 may be a wired or wireless network, or any variety of other communication links. Communication channel 600 carries signals 605 and can be implemented using a variety of wired or wireless communication means including wire or cable, fiber optics, conventional phone line, cellular phone link, wireless data communication link, radio frequency (“RF”) link, or infrared link, just to name a few.

Computer executable code (i.e., computer programs or software) is stored in the main memory 565 and/or the secondary memory 570. Computer programs can also be received via communication interface 590 and stored in the main memory 565 and/or the secondary memory 570. Such computer programs, when executed, enable the system 550 to perform the various functions of the present invention as previously described.

In this description, the term “computer readable medium” is used to refer to any non-transitory computer readable storage media used to provide computer executable code (e.g., software and computer programs) to the system 550. Examples of these media include main memory 565, secondary memory 570 (including internal memory 575, removable medium 580, and external storage medium 595), and any peripheral device communicatively coupled with communication interface 590 (including a network information server or other network device). These non-transitory computer readable mediums are means for providing executable code, programming instructions, and software to the system 550.

In an embodiment that is implemented using software, the software may be stored on a computer readable medium and loaded into the system 550 by way of removable medium 580, I/O interface 585, or communication interface 590. In such an embodiment, the software is loaded into the system 550 in the form of electrical communication signals 605. The software, when executed by the processor 560, preferably causes the processor 560 to perform the inventive features and functions previously described herein.

The system 550 also includes optional wireless communication components that facilitate wireless communication over a voice and over a data network. The wireless communication components comprise an antenna system 610, a radio system 615 and a baseband system 620. In the system 550, radio frequency (“RF”) signals are transmitted and received over the air by the antenna system 610 under the management of the radio system 615.

In one embodiment, the antenna system 610 may comprise one or more antennae and one or more multiplexors (not shown) that perform a switching function to provide the antenna system 610 with transmit and receive signal paths. In the receive path, received RF signals can be coupled from a multiplexor to a low noise amplifier (not shown) that amplifies the received RF signal and sends the amplified signal to the radio system 615.

In alternative embodiments, the radio system 615 may comprise one or more radios that are configured to communicate over various frequencies. In one embodiment, the radio system 615 may combine a demodulator (not shown) and modulator (not shown) in one integrated circuit (“IC”). The demodulator and modulator can also be separate components. In the incoming path, the demodulator strips away the RF carrier signal leaving a baseband receive audio signal, which is sent from the radio system 615 to the baseband system 620.

If the received signal contains audio information, then baseband system 620 decodes the signal and converts it to an analog signal. Then the signal is amplified and sent to a speaker. The baseband system 620 also receives analog audio signals from a microphone. These analog audio signals are converted to digital signals and encoded by the baseband system 620. The baseband system 620 also codes the digital signals for transmission and generates a baseband transmit audio signal that is routed to the modulator portion of the radio system 615. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 610 where the signal is switched to the antenna port for transmission.

The baseband system 620 is also communicatively coupled with the processor 560. The central processing unit 560 has access to data storage areas 565 and 570. The central processing unit 560 is preferably configured to execute instructions (i.e., computer programs or software) that can be stored in the memory 565 or the secondary memory 570. Computer programs can also be received from the baseband processor 610 and stored in the data storage area 565 or in secondary memory 570, or executed upon receipt. Such computer programs, when executed, enable the system 550 to perform the various functions of the present invention as previously described. For example, data storage areas 565 may include various software modules (not shown) that are executable by processor 560.

Various embodiments may also be implemented primarily in hardware using, for example, components such as application specific integrated circuits (“ASICs”), or field programmable gate arrays (“FPGAs”). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, and methods described in connection with the embodiments disclosed herein can be implemented or performed with a general purpose processor, a digital signal processor (“DSP”), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such the processor can read information from, and write information to, the storage medium. In the alternative, the storage medium can be integral to the processor. The processor and the storage medium can also reside in an ASIC.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

1. An apparatus, comprising:
an explosive trace detection (ETD) unit;
a nuclear quadrupole resonance (NQR) spectroscopy unit;
a user interface module configured to present, on a display, an alarm indication in response to at least one of the following:
a detection of a presence of a first explosive by the ETD unit; and
a detection of a presence of the first explosive or a second explosive by the NQR spectroscopy unit.

2. The apparatus of claim 1, further comprising a radio frequency (RF) shield.

3. The apparatus of claim 1, further comprising an air containment chamber disposed inside the RF shield.

4. (canceled)

5. The apparatus of claim 1, further comprising a coupling device configured to interface the apparatus with the ETD unit and at least one other type of ETD unit.

6. The apparatus of claim 3, further comprising a door at an opening into the air containment chamber.

7. The apparatus of claim 3, further comprising a tray that is configured to slide in and out of the air containment chamber.

8. The apparatus of claim 3, further comprising a conveyor system.

9. The apparatus of claim 3, wherein the air containment chamber is configured to provide a hermetically sealed environment.

10. The apparatus of claim 1, wherein the NQR spectroscopy unit comprises an RF antenna.

11. The apparatus of claim 9, wherein the RF antenna is disposed inside the air containment chamber, and is configured to permit at least one of the following:
an entry of one or more gaseous substances into the air containment chamber; and
an exit of one or more gaseous substances from the air containment chamber.

12. The apparatus of any of claim 11, wherein the RF antenna is disposed outside of the air containment chamber.

13. A method, comprising:
placing an object in the air containment chamber of the apparatus of claim 1;
performing explosive trace detection (ETD) on the object;
performing nuclear quadrupole resonance (NQR) spectroscopy on the object;
analyzing the results from the ETD the NQR spectroscopy; and
detecting a presence of at least one explosive based on the analysis of the results from the ETD and the NQR spectroscopy performed on the object.

14. The method of claim 13, wherein the ETD and the NQR spectroscopy are performed simultaneously.

15. The method of claim 13, wherein the ETD and the NQR spectroscopy are performed sequentially.

16. The method of claim 13, further comprising electromagnetically shielding the air containment chamber of the apparatus of claim 1.

17. The method of claim 13, further comprising hermetically sealing the air containment chamber of the apparatus of claim 1.

18. The method of claim 13, further comprising generating an alarm in response to at least one of the following:
a detection of a presence of a first explosive based on the analysis of the result from the ETD; and
a detection of a presence of the first explosive or a second explosive based on the analysis of the result from the NQR spectroscopy.

19. The method of claim 13, wherein performing the ETD comprises exposing the object to one or more gaseous substances inside the air containment chamber, and analyzing at least one air sample extracted from the air containment chamber subsequent to exposing the object to the one or more gaseous substances.

20. The method of claim 13, wherein performing the NQR spectroscopy comprises:
applying interrogation electromagnetic radiation to the object at a first frequency corresponding to an NQR frequency of a first explosive; and
measuring feedback electromagnetic radiation emitted by the object in response to the interrogation electromagnetic radiation at the first frequency.

21. The method of claim 13, further comprising:
repeating a performance of at least one of ETD and NQR spectroscopy on the object;
analyzing a result from the at least one of the ETD and the NQR spectroscopy repeated on the object; and
detecting a presence of at least one explosive based on the analysis of the results from the at least one of the ETD and the NQR spectroscopy repeated on the object.
